(19) United States
(12) Patent Application Publication
(10) Pub. No.: US 2017/0056248 A1
(43) Pub. Date: Mar. 2, 2017

(54) AudioOptics

(71) Applicant: James Duncan Lowe, San Rafael, CA (US)

(72) Inventor: James Duncan Lowe, San Rafael, CA (US)

(21) Appl. No.: 14/840,697

(22) Filed: Aug. 31, 2015

(51) Int. Cl. A61F 11/00; G02C 11/06; G10K 11/28

(52) U.S. Cl. CPC A61F 11/008; G10K 11/28; G02C 11/06

(57) ABSTRACT

This invention relates to a prescription eyeglass mounted sound reflective hearing aid device with sound reflectors that remove surrounding noise. The hearing aid device is comprised of a pair of eyeglasses with two molded half cone shaped directional sound reflectors. Both of the reflectors have an interior surface and an exterior surface. The reflectors take surrounding noise out of the wearer's perception, making it easier to listen to people.

### CROSS REFERENCE TO RELATED APPLICATIONS

Not applicable

### FEDERALLY SPONSORED RESEARCH AND DEVELOPMENT

Not applicable.

### MICROFICHE

Not applicable

### BACKGROUND OF THE INVENTION

(1) Field of the Invention

The present invention relates to the field of hearing aid devices. More particularly, the present invention relates to a prescription eyeglass mounted sound reflective hearing aid device with sound reflectors that remove surrounding noise.

(2) Background of the Invention

Prescription eyeglass mounted hearing aid devices are well known in the art. Although there are electronic means for amplifying sound waves for a hearing-impaired person, the electronics that are utilized often introduce extraneous background noise along with the intended signal for amplification. Electronic devices are also expensive for occasional use by persons having normal hearing capabilities. Hearing horns or trumpets were developed very early, but tended to produce a propagation of standing waves in the chamber, causing distortions to the sound waves amplified. These devices are generally inserted into the ear canal and are cumbersome to use. Later devices introduced reflectors intended to either focus sound waves into the ear, or provide a larger collection area for sound waves.

U.S. Pat. No. 3,247,330 describes a hearing aid instrument that is adapted to provide stereophonic hearing, which is as close to natural hearing as is possible. The hearing aid instrument is arranged on the head of the user so that the sound detector is shielded from direct sound waves, and is adapted to only receive sound waves that are reflected from the face of the user, generally between the eye socket and the cheek.

U.S. Pat. No. 6,690,807 discloses a simple and low cost listening assistance device that can be carried without any problems, is inconspicuous, and specifically counteracts the reduced understanding of spoken matter. The listening assistance device includes angled spectacle frame temples, which lead to a specifically good positioning of the spectacles, excellent wearing comfort, and an inconspicuous appearance.

U.S. Pat. App. No. 20080123884 describes a passive hearing aid device that mechanically receives a greater amount of directional acoustic waves than an unaided ear. The device mechanically reflects sound waves into the user's ear or ears without distortion. The invention provides a means for reducing interference from background noise through the utilization of sound damping materials in the construction of the device.

U.S. Pat. No. 8,107,660 discloses a hearing aid that can prevent the output of a sound amplified by the hearing aid processing before the user attaches the hearing aid. It is able to do this even if it takes time between turning on the hearing aid and attaching the hearing aid.

Several of these inventions describe an apparatus for either focusing or collecting sound waves, and improve the comfort of the attachment of the device to the user. One even incorporates the utilization of sound reflectors that take noise out of the wearer's perception, so as to ease listening. What is needed is a hearing aid device that provides improved sound wave collection and incorporates sound reflectors to reflect sound into the ear canal without using a battery.

Thus, there is a long-felt need to provide a prescription eyeglass mounted hearing aid device with directional sound reflectors that works by reflecting sound coming from in front of wearer into the ear canal.

The foregoing patents and prior art devices reflect the current state of the art, of which the present inventor is aware. Reference to, and discussion of, these patents is intended to aid in discharging the applicant's acknowledged duty of candor in disclosing information that may be relevant to the examination of prospective claims to the present invention. However, it is respectfully submitted that none of the above-indicated patents disclose, teach, suggest, show, or otherwise render obvious, either singly or when considered in combination, the invention described herein.

### SUMMARY OF THE INVENTION

In the view of the foregoing disadvantages inherent in the known types of hearing aid devices now present in the prior art, the present invention provides an eyeglass mounted sound reflective hearing aid device. As such, the general purpose of the present invention, which will be described subsequently in greater detail, is to provide an eyeglass mounted sound reflective hearing aid device that has all of the advantages of the prior art, and none of the disadvantages.

An objective of the invention is to provide a prescription eyeglasses mounted sound reflective hearing aid device with sound reflectors that removes surrounding noise.

It is another objective of the present invention to provide a hearing aid device that allows for easier conversation in high ambient sound situations, such as at the beach, waterfalls, crowded sporting events, etc.

It is further an objective of the present invention to provide a hearing aid device that works without batteries.

It is another objective of the present invention to provide a hearing device that needs no adjustments.

It is another objective of the present invention to provide a hearing aid device that does not require periodic check-ups for updates.

It is yet another objective of the present invention to provide a hearing aid device that can be used in any environment.

It is yet another objective of the present invention to provide a hearing aid device that can be easily adjusted by the wearer to work for any ear shape or size.

It is yet another objective of the present invention to provide snap on sound reflectors that can be made to fit any prescription glasses.

It is still another objective of the present invention to provide a hearing aid device that can be used by those without hearing loss.

It is still another objective of the present invention to provide a hearing aid device that can be manufactured very inexpensively.

It is further an objective of the present invention to provide a hearing aid device that reflects sound into the ear canal.

In this respect, before explaining at least one embodiment of the invention in detail, it is to be understood that the invention is not limited in its application to the details of construction and to the arrangements of the components set forth in the following description or illustrated in the drawings. The invention is capable of other embodiments, and of being practiced and carried out in various ways. Also, it is to be understood that the phraseology and terminology employed herein are for the purpose of description, and should not be regarded as limiting.

These, together with other objectives of the invention and the various features of novelty that characterize the invention, are pointed out with particularity in the disclosure. For a better understanding of the invention, its operating advantages, and the specific objectives attained by its uses, reference should be had to the accompanying drawings and descriptive matter, in which there are illustrated preferred embodiments of the invention.

### BRIEF DESCRIPTION OF TILE DRAWINGS

The invention will be better understood, and objectives other than those set forth above will become apparent, when consideration is given to the following detailed description thereof. Such description makes reference to the annexed drawings, wherein:

FIG. 1 shows the preferred embodiment of a pair of eyeglasses with two molded reflectors.

### DETAILED DESCRIPTION OF THE INVENTION

In the following detailed description, reference is made to the accompanying drawings, which form a part hereof, and in which is shown, by way of illustration, specific embodiments in which the invention may be practiced. These embodiments are described in sufficient detail to enable those skilled in the art to practice the invention, and it is to be understood that the embodiments may be combined, or that other embodiments may be utilized, and that structural, logical, and electrical changes may be made without departing from the spirit and scope of the present invention. The following detailed description is, therefore, not to be taken in a limiting sense, and the scope of the present invention is defined by the appended claims and their equivalents.

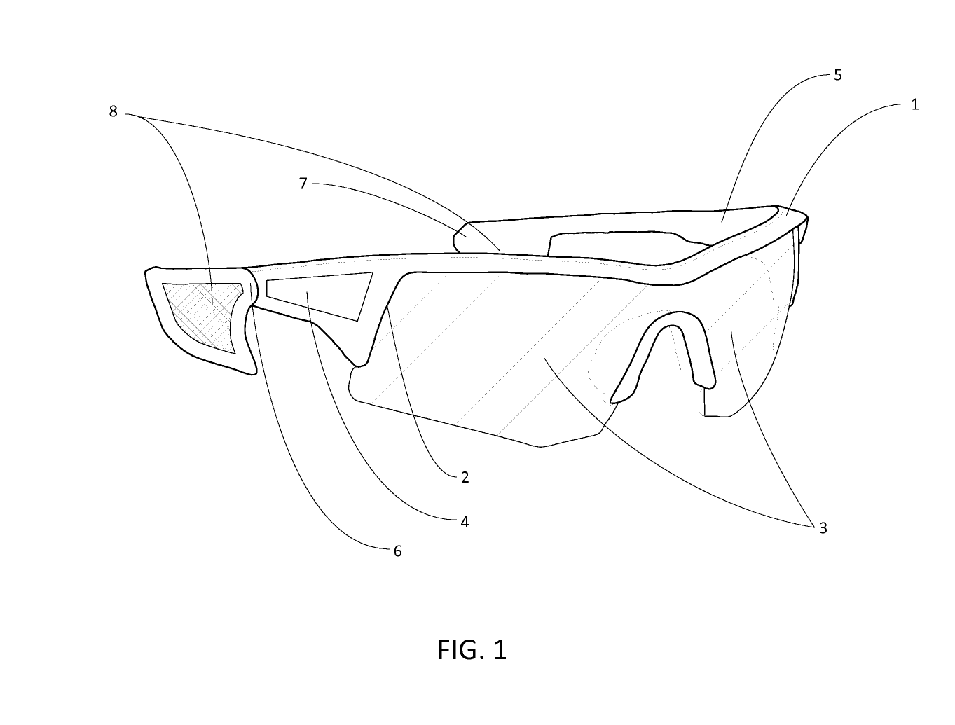

FIG. 1 illustrates a pair of prescription eyeglasses that are designated by the numeral. The eyeglasses include a frame 1 that may be made in various designs and from various types of plastic or other suitable materials. The component parts of the frame are of substantial dimensions in accordance with the present-day designs, thus making the combining of the hearing aid equipment with the frame readily possible. The eyeglass frame has the usual cross-bow member 2, which provides a pair of oval rims that are integrally formed in one piece. The rims enclose or surround the lenses 3 in the usual manner. Secured to the crossbow, either rigidly, slightly flexible, or pivotally connected thereto, are the templar members or temple bars 4 and 5. They extend rearwardly therefrom. The templar members extend adjacent to the side of the head of the user, and are provided with the customary curved portions 6 and 7 respectively, which are adapted to rest on the ears. The curved portions serve as ear mounts.

The side temple members 4 and 5 are of tubular construction and are generally made in one piece, so as to provide a solid bar type shape. The cross-bow is provided with the usual bridge, which connects the rims, and is adapted to rest on the nose of the user when the frame is mounted properly on the head of the user in a substantially fixed position.

The snap connectors are pivotally connected to a pair of directional sound reflectors 8. Both of the sound reflectors are each shaped in the form of a half cone. Each has an interior surface and an exterior surface that form a “C” shaped interface surface surrounding the rear portion of the user's ear. This construction leaves the front of the ear exposed for receiving sound.

The invention works by reflecting sound coming from in front of wearer into the ear canal. The interior surface of both of the directional sound reflectors reflects the sound into the ear canal. With this device, the wearer cannot only hear what is being said by those in front of him, but himself as well. The device does not require any battery or wiring. It does not require to be inserted into the ear.

The present invention is primarily directed towards enhancing the listening capabilities of a user at the beach, waterfalls, crowded sporting events, or the like, where the auditory signals are not focused towards the listener. However, the invention may be employed to enhance the auditory experience of a user in other listening environments as well.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages that may be provided by the present invention have been described above with regard to specific embodiments. These benefits and advantages, and any elements or limitations that may cause them to occur or to become more pronounced, are not to be construed as critical, required, or essential features of any or all of the embodiments.

While the present invention has been described with reference to particular embodiments, it should be understood that the embodiments are illustrative, and that the scope of the invention is not limited to these embodiments. Many variations, modifications, additions, and improvements to the embodiments described above are possible. It is contemplated that these variations, modifications, additions, and improvements fall within the scope of the invention.

I claim:

1. A hearing aid structure, comprising:
an eyeglass frame;
at least one directional sound reflector; and
at least one pivotal connector.

2. The hearing aid structure of claim 1, wherein said eyeglass frame is adapted to be worn in a substantially fixed position on the head of a user.

3. The hearing aid structure of claim 1, wherein said at least one directional sound reflector has an interior surface and an exterior surface.

4. The hearing aid structure of claim 1, wherein said at least one directional sound reflector takes the surrounding noise out of a user's perception.

5. The hearing aid structure of claim 1, wherein said eyeglass frame further comprising a cross-bow member and a pair of side templar members attached to said cross-bow member.

6. The hearing aid structure of claim 5, wherein said cross-bow member defines the rims for retaining a pair of lenses.

7. The hearing aid structure of claim 5, wherein said pair of side templar members are provided with curved portions at their rear end.

8. The hearing aid structure of claim 5, wherein each curved portion of said pair of side templar members is attached to said at least one pivotal connector.

9. The hearing aid structure of claim 5, wherein said at least one pivotal connector is connected to at least one directional sound reflector.

10. The hearing aid structure of claim 9, wherein the interior surface of said at least one directional sound reflector reflects sound into ear canal.
